(19) United States
(12) Patent Application Publication
(10) Pub. No.: US 2025/0177209 A1
(43) Pub. Date: Jun. 5, 2025

(54) Protection for direct selective laser trabeculoplasty

(71) Applicant: Belkin Vision Ltd., Yavne (IL)

(72) Inventors: Zachary Sacks, Modiin (IL); Daria Lemann-Blumenthal, Bitzaron (IL)

(21) Appl. No.: 19/051,279

(22) Filed: Feb. 12, 2025

Related U.S. Application Data
(62) Division of application No. 17/273,323, filed on Mar. 4, 2021.
(60) Provisional application No. 62/751,629, filed on Oct. 28, 2018.

(51) Int. Cl. A61F 9/008; A61F 9/009

(52) U.S. Cl. CPC A61F 9/0084; A61F 9/009; A61F 2009/00846; A61F 2009/00868; A61F 2009/00891

(57) ABSTRACT

A system for treating an eye of a patient includes a camera, configured to acquire an image of the eye, and a radiation source. A controller is configured to identify a static region in a field of view of the camera that, in the image, is outside a limbus of the eye, and to treat one or more target regions of the eye, by, for each target region ascertaining that the target region is not within the static region, and in response to the ascertaining, causing the radiation source to irradiate the target region.

### CROSS-REFERENCE TO RELATED APPLICATIONS

The present application is a division of U.S. patent application Ser. No. 17/273,323, filed Mar. 4, 2021, in the national phase of PCT Patent Application PCT/IB2019/059058, filed Oct. 23, 2019, which claims the benefit of U.S. Provisional Appl. No. 62/751,629, entitled “DSLT Eye Protection,” filed Oct. 28, 2018, whose disclosure is incorporated herein by reference.

### FIELD OF THE INVENTION

The present invention relates to ophthalmological devices and methods for the treatment of glaucoma, ocular hypertension (OHT), and other diseases.

### BACKGROUND

In a trabeculoplasty procedure, a radiation source, typically a laser, irradiates the trabecular meshwork in an eye of a patient with one or more treatment beams, thus lowering the intraocular pressure in the eye.

U.S. Pat. No. 4,848,894 describes a contact lens made either with a laser-reflecting or absorbing layer embedded in a transparent optical lens material, or formed as a layer on the convex side of such a material. The layer may be a Fabry-Perot reflector or a thin-film or holographically formed reflective or absorptive interference filter, or an absorbing layer.

U.S. Pat. No. 4,966,452 describes a contact lens for use in connection with transscleral cyclophotocoagulation. The lens has a planar entry surface and a frustoconically-shaped exit surface that contacts the sclera surrounding the cornea. The central portion of the lens is opaque to prevent stray laser light from entering the optical portion of the eye during laser application.

U.S. Pat. No. 6,948,815 describes individually marked contact lenses for protection of one's eyes from harmful radiation. Contact lenses are coated or treated to be absorptive or reflective to a preselected wavelength or wavelengths. The lenses contain one or more identification areas on each lens to demonstrate that the lenses are being worn and to indicate the proper applications with which the lenses should be used and/or the wavelengths for which the lens is protective. The identification area, which should be visible when worn to third parties and/or the person wearing the lenses, consists of markings such as colored bands or shaded areas in the region around the iris. Different colors or color patterns of the markings indicate which wavelengths the lens protects against. Other safety features may include fluorescent markers, added features and devices to facilitate placement and retention in the eye, pick-up or release.

U.S. Pat. No. 8,004,764 describes a filter and method for filtering an optical beam. One embodiment of the filter is an optical filter for filtering an incident light beam, comprising an optically effective material characterized by: a light transmittance of less than 1% for wavelengths below 420 nm; and a light transmittance for wavelengths complementary and near complementary to wavelengths below 420 nm that, combined with the transmittance for wavelengths below 420 nm, will yield a filtered light beam having a luminosity of about 90% and an excitation purity of 5% or less. The complementary wavelengths can be wavelengths above about 640 nm, wavelengths above about 660 nm, and/or wavelengths from about 540 nm to about 560 nm.

U.S. Pat. No. 9,480,599 describes an ophthalmic laser ablation system with various optional features, some especially suitable for non-penetrating filtration surgery on an eye. In one example, focusing of an ablation laser uses a movable lens coupled to a pair of converging light sources, which converge at the focal distance of the lens. In another example, laser ablation settings are selected for optimal ablation and minimal amount of thermal damage of a layer of percolating scleral tissue.

International Patent Application Publication WO/1998/022016 describes a combination of a scanning laser ophthalmoscope and a photocoagulator. The device includes a therapeutic laser source, optic-mechanical Maxwellian view coupling, Maxwellian view control, and real-time electronic registration of the therapeutic beam location.

### SUMMARY OF THE INVENTION

There is provided, in accordance with some embodiments of the present invention, an apparatus including an optical unit. The optical unit includes a light source, one or more beam-directing elements, and a radiation source. The radiation source is configured to irradiate an eye of a patient with one or more treatment beams by emitting the treatment beams toward the beam-directing elements, while the eye fixates on the light source by virtue of the light source transmitting visible light. The apparatus further includes an optical filter configured to inhibit passage of the treatment beams, but not the visible light, therethrough, while interposing between the beam-directing elements and a pupil of the eye.

In some embodiments, the apparatus further includes an eye-stabilizing structure, the radiation source is configured to irradiate the eye by emitting the treatment beams through the structure, and the optical filter is coupled to the structure.

In some embodiments, a proximal end of the structure is configured to couple to the optical unit.

In some embodiments, the optical filter is coupled to a distal end of the structure.

In some embodiments, the apparatus further includes a contact optic including the optical filter, and the contact optic is coupled to the distal end of the structure and is configured to contact the eye.

In some embodiments, a distal end of the structure is configured to contact the eye, and the optical filter is mounted to an inner wall of the structure.

In some embodiments, the optical filter is mounted between 0.5 and 20 mm from the distal end of the structure.

In some embodiments, the distal end of the structure includes a contact optic configured to contact the eye.

In some embodiments, the distal end of the structure includes a contact ring configured to contact the eye.

In some embodiments, the apparatus further includes one or more longitudinal elements extending between the inner wall of the structure and the optical filter, the optical filter being mounted to the inner wall via the longitudinal elements.

In some embodiments, an inner wall of the structure is configured to absorb the treatment beams.

In some embodiments, a treatment-beam wavelength of the treatment beams is between 200 and 11000 nm, and a visible-light wavelength of the visible light is between 350 and 850 nm.

In some embodiments, the optical filter is configured to inhibit the passage of the treatment beams by absorbing the treatment beams.

In some embodiments, the optical filter is configured to inhibit the passage of the treatment beams by reflecting the treatment beams.

In some embodiments, the optical filter includes an anti-reflective surface configured to reduce reflection of the treatment beams.

In some embodiments, the apparatus further includes an optic including:
the optical filter; and
a transparent portion that surrounds the optical filter and is transparent to the treatment beams.

In some embodiments,
the optical filter is a first optical filter, and
the optic further includes a second optical filter that surrounds the transparent portion and is configured to inhibit the passage of the treatment beams.

In some embodiments, the apparatus further includes a contact optic including the optical filter, and the contact optic is configured to contact the eye.

In some embodiments, a thickness of the contact optic is less than 2 mm.

In some embodiments, a diameter of the contact optic is less than 10 mm.

In some embodiments,
the optical unit includes a front face, and
the optical filter is coupled to the front face.

In some embodiments,
the front face includes an exit window,
the light source is configured to transmit the visible light, and the radiation source is configured to emit the treatment beams, through the exit window, and
the optical filter overlays the exit window.

In some embodiments,
the front face includes an exit window,
the light source is configured to transmit the visible light, and the radiation source is configured to emit the treatment beams, through the exit window, and
the optical filter is embedded within the exit window.

In some embodiments, the apparatus further includes a longitudinal element extending between the front face and the optical filter, the optical filter being coupled to the front face via the longitudinal element.

In some embodiments,
the front face includes an exit window,
the light source is configured to transmit the visible light, and the radiation source is configured to emit the treatment beams, through the exit window, and
the longitudinal element extends between the exit window and the optical filter.

There is further provided, in accordance with some embodiments of the present invention, a method including interposing an optical filter between one or more beam-directing elements and a pupil of an eye of a patient. The method further includes, while the optical filter interposes between the beam-directing elements and the pupil, using a light source, transmitting visible light through the optical filter and, while the eye fixates on the light source by virtue of the light source transmitting the visible light, irradiating the eye with one or more treatment beams by emitting the treatment beams toward the beam-directing elements, the optical filter being configured to inhibit passage of the treatment beams therethrough.

In some embodiments, the optical filter belongs to a contact optic, and interposing the optical filter includes interposing the optical filter by contacting the eye with the contact optic.

There is further provided, in accordance with some embodiments of the present invention, a system including a camera, configured to acquire an image of an eye of a patient, a radiation source, and a controller. The controller is configured to identify a static region in a field of view of the camera that, in the image, is outside a limbus of the eye, and to treat one or more target regions of the eye, by, for each target region, ascertaining that the target region is not within the static region, and in response to the ascertaining, causing the radiation source to irradiate the target region.

There is further provided, in accordance with some embodiments of the present invention, a method including, using a camera, acquiring an image of an eye of a patient. The method further includes identifying a static region in a field of view of the camera that, in the image, is outside a limbus of the eye. The method further includes treating one or more target regions of the eye, by, for each target region, ascertaining that the target region is not within the static region, and in response to the ascertaining, irradiating the target region.

There is further provided, in accordance with some embodiments of the present invention, an apparatus including an optical unit. The optical unit includes a light source, configured to transmit visible light, one or more beam-directing elements, and a radiation source, configured to irradiate a first eye of a patient with one or more treatment beams by emitting the treatment beams toward the beam-directing elements such that the beam-directing elements direct the treatment beams along an optical path to the first eye. The apparatus further includes an optical filter configured to inhibit the visible light and the treatment beams from reaching a pupil of the first eye. The light source is displaced from the optical path towards a second eye of the patient, and the radiation source is configured to irradiate the first eye while the second eye fixates on the light source by virtue of the light source transmitting the visible light.

In some embodiments, the light source is displaced from the optical path by 18-44 mm.

There is further provided, in accordance with some embodiments of the present invention, a method, including interposing an optical filter, which is opaque to visible light transmitted by a light source and to treatment beams emitted by a radiation source, between (i) one or more beam-directing elements and (ii) a pupil of a first eye of a patient. The method further includes, while a second eye of the patient fixates on the light source by virtue of the light source transmitting the visible light, using the radiation source, irradiating the first eye with the treatment beams by emitting the treatment beams toward the beam-directing elements.

In some embodiments, the beam-directing elements direct the treatment beams along an optical path to the first eye, and the method further includes, prior to irradiating the first eye, displacing the light source from the optical path towards the second eye.

The present invention will be more fully understood from the following detailed description of embodiments thereof, taken together with the drawings, in which:

### BRIEF DESCRIPTION OF THE DRAWINGS

FIG. 1 is a schematic illustration of a system for performing a trabeculoplasty procedure, in accordance with some embodiments of the present invention;

FIG. 2 is a schematic illustration of a trabeculoplasty device, in accordance with some embodiments of the present invention;

FIGS. 3A-B are schematic illustrations of an eye-stabilizing structure as viewed from the side and from the front, respectively, in accordance with some embodiments of the present invention;

FIGS. 4A-B are schematic illustrations of an eye-stabilizing structure as viewed from the side and from the front, respectively, in accordance with some embodiments of the present invention;

FIGS. 5-6 are schematic illustrations of an optical filter coupled to an optical unit, in accordance with some embodiments of the present invention;

FIG. 7 is a schematic illustration of a technique for orienting an eye for treatment, in accordance with some embodiments of the present invention; and

FIG. 8 is a schematic illustration of an image of an eye treated in accordance with some embodiments of the present invention.

### DETAILED DESCRIPTION OF EMBODIMENTS

### Overview

Embodiments of the present invention provide an automated trabeculoplasty device configured to perform a trabeculoplasty procedure on an eye safely and efficiently. The trabeculoplasty device comprises a controller and an optical unit, which comprises a radiation source (typically a laser), a camera and one or more illumination sources for imaging, one or more beam-directing elements, and a light source. By transmitting (i.e., emitting or reflecting) visible light, the light source functions as a fixation target for the eye. While the eye fixates on the light source, the controller, in response to feedback from the camera, causes the beam-directing elements to direct beams of radiation, which are emitted by the radiation source, toward targeted regions of the eye, which are typically located in the vicinity of the limbus of the eye.

Embodiments of the present invention further provide an optical filter that is opaque to the treatment beams, but not to the visible light transmitted by the light source. By interposing between the optical unit and the pupil of the eye, the optical filter protects the retina of the eye from any stray radiation beams without inhibiting the eye from fixating on the fixation target.

In some embodiments, the optical filter belongs to a contact optic, which is worn in the eye without being held in place by any other element of the system. In other embodiments, the optical filter is mounted within or at the end of an eye-stabilizing structure, such as a hollow frustum-shaped structure, extending between the optical unit and the eye. In addition to supporting the optical filter, the eye-stabilizing structure may perform additional functions; for example, the eye-stabilizing structure may hold the eye open, stabilize the eye, and/or absorb any misaimed treatment beams. In yet other embodiments, the optical filter overlays the exit window of the optical unit, is embedded within the exit window, or is otherwise coupled to the optical unit.

In some embodiments, the peripheral portion of the eye lying outside the target regions is also protected. This protection may be provided by an additional (physical) optical filter. Alternatively or additionally, this protection may be provided by a virtual filter, in that the controller may identify a region in the camera's FOV in which the peripheral portion of the eye is located, and then inhibit the treatment beams from being fired at this region.

Although the present description focuses mainly on trabeculoplasty procedures, the techniques described herein may also be applied to automatic photocoagulation procedures, iridotomy procedures, corneal procedures, capsulectomy procedures, lens removals, or any other relevant ophthalmological procedures. The target of the radiation may include the trabecular meshwork and/or any other suitable portion of the eye. Embodiments of the present invention may be used to treat glaucoma, ocular hypertension (OHT), and other diseases.

It is noted that in the context of the present application, including the claims, an optical element is said to be “opaque” to visible light transmitted therethrough if the element attenuates the light (by reflection and/or absorption) to an intensity that is less than the minimum intensity at which an average human eye is able to perceive the light. For other types of radiation, the optical element is said to be opaque to the radiation if the element attenuates the radiation (by reflection and/or absorption) to an intensity that is less than the maximum permissible exposure (MPE) value for the radiation as defined in any relevant standard, such as the International Electrotechnical Commission (IEC) 60825 standard.

Conversely, in the context of the present application, including the claims, an optical element is said to be “transparent” to radiation if the element is not opaque to the radiation.

### System Description

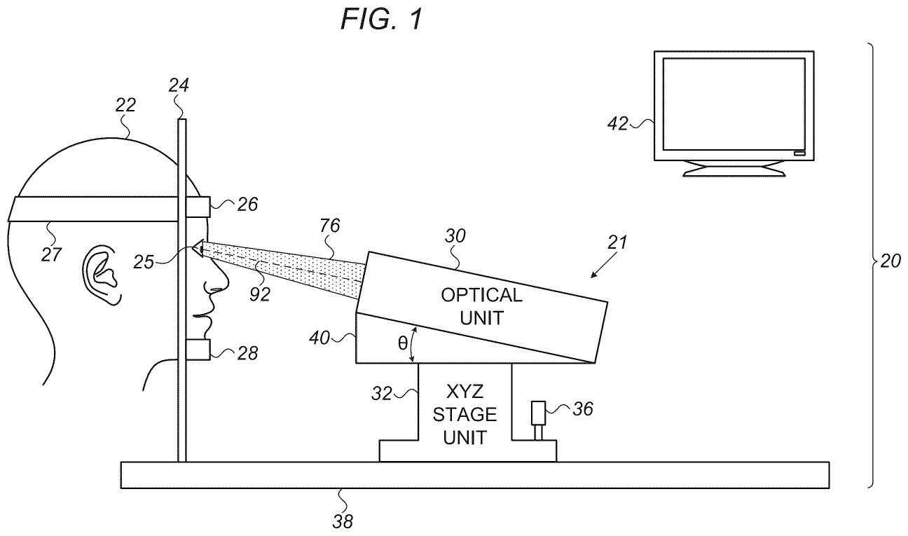

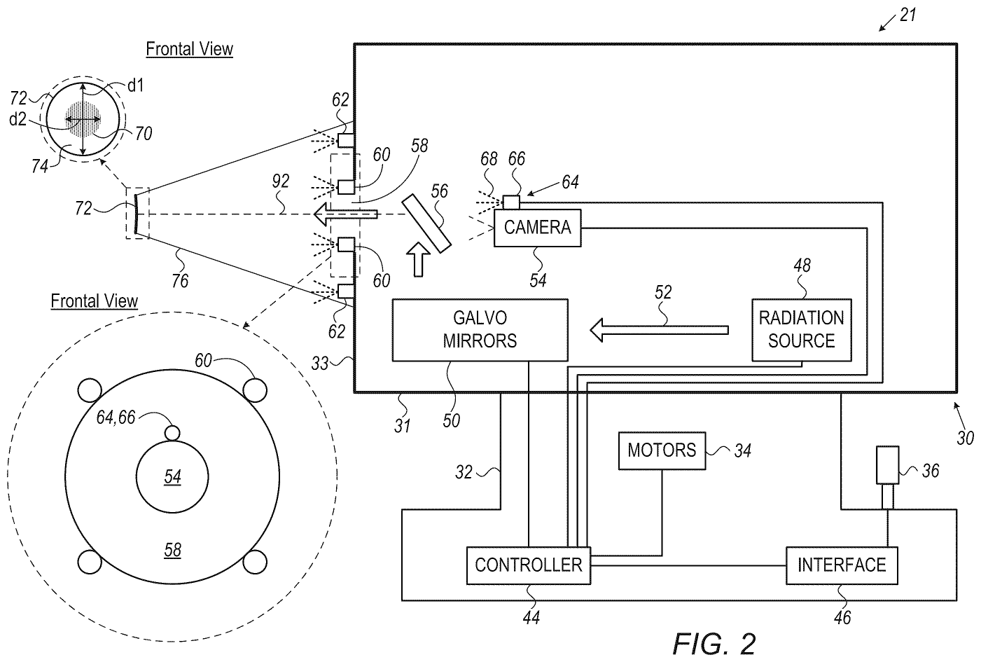

Reference is initially made to FIG. 1, which is a schematic illustration of a system 20, comprising a trabeculoplasty device 21, for performing a trabeculoplasty procedure, in accordance with some embodiments of the present invention. Reference is further made to FIG. 2, which is a schematic illustration of trabeculoplasty device 21, in accordance with some embodiments of the present invention.

Trabeculoplasty device 21 comprises an optical unit 30 and a controller 44. Optical unit 30 comprises one or more beam-directing elements, comprising, for example, one or more galvo mirrors 50, which may be referred to collectively as a “galvo scanner,” and/or a beam combiner 56. Optical unit 30 further comprises a radiation source 48, which is configured to irradiate an eye 25 of a patient 22 with one or more treatment beams 52 by emitting the treatment beams toward the beam-directing elements such that the beams are directed by the beam-directing elements toward the eye.

More specifically, before the emission of each treatment beam 52 from radiation source 48, or while the beam is being emitted, controller 44 aims the beam-directing elements at the desired target region on eye 25 such that the beam is directed, by the beam-directing elements, toward the target region. For example, the beam may be deflected by galvo mirrors 50 toward beam combiner 56, and then deflected by the beam combiner such that the beam impinges on the target region. (Since each treatment beam impinges on the eye with a non-infinitesimal spot size, the present application generally describes each beam as impinging on a “region” of the eye, whose area is a function of the spot size, rather than impinging at a “point” on the eye.) The beam thus follows an optical path 92, which extends from the most downstream of the beam-directing elements—such as beam combiner 56—to eye 25. (In practice, the respective paths of the beams vary slightly from each other due to the small distances between the target regions; however, given that this variation is very slight, the present description refers to a single optical path 92 followed by all the treatment beams.)

Typically, the radiation source comprises a laser, such as an Ekspla™ NL204-0.5K-SH laser. The laser may be modified to include an attenuator, an energy meter, and/or a mechanical shutter. Alternatively or additionally to a laser, the radiation source may comprise any other suitable emitter.

In some embodiments, the treatment beams comprise visible light. Alternatively or additionally, the treatment beams may comprise non-visible radiation, such as microwave radiation, infrared radiation, X-ray radiation, gamma radiation, or ultraviolet radiation. Typically, the wavelength of the treatment beams is between 200 and 11000 nm, e.g., 500-850 nm, such as 520-540 nm, e.g., 532 nm. Each treatment beam 52 may have an elliptical (e.g., a circular) shape, a square shape, or any other suitable shape.

Optical unit 30 further comprises a camera 54. As shown in FIG. 2, camera 54 is typically aligned, at least approximately, with optical path 92; for example, the angle between optical path 92 and a hypothetical line extending from eye 25 to the camera may be less than 15 degrees. In some embodiments, the camera is positioned behind beam combiner 56, such that the camera receives light via the beam combiner.

Before the procedure, camera 54 acquires at least one image of eye 25. Based on the image, controller 44 and/or a user of the system, such as an ophthalmologist or another physician, may define and/or modify the target regions of the eye that are to be irradiated. Alternatively or additionally, based on the image, controller 44 may define one or more virtual filters, as further described below with reference to FIG. 8. Subsequently, during the procedure, camera 54 may acquire multiple images of the patient's eye at a relatively high frequency. Controller 44 may process these images and, in response thereto, control radiation source 48 and the beam-directing elements so as to irradiate the target regions of the eye.

Optical unit 30 further comprises a light source 66, which is aligned, at least approximately, with optical path 92. For example, the angle between optical path 92 and a hypothetical line extending from the end of path 92 on eye 25 to light source 66 may be less than 20 degrees, such as less than 10 degrees. Light source 66 is configured to function as a fixation target 64 by transmitting visible light 68. (It is noted that visible light 68 may also be described as a “fixation beam” or as “fixation light.”)

In particular, prior to the procedure, patient 22 is instructed to fixate eye 25 on light source 66. Subsequently, during the procedure, by virtue of light source 66 transmitting visible light 68, eye 25 fixates on the light source, such that the eye's line-of-sight is approximately coincident with optical path 92 (due to the light source being approximately aligned with the optical path) and the eye is relatively stable. While the eye fixates on the light source, the radiation source irradiates the eye with treatment beams 52. Light source 66 may also help orient the eye while an aiming beam is swept across the eye prior to the procedure, as further described below with reference to FIG. 8.

In some embodiments, light source 66 comprises a light emitter, such as a light emitting diode (LED). In other embodiments, the light source comprises a reflector configured to reflect light emitted from a light emitter.

Typically, the wavelength of visible light 68, which may be higher or lower than that of the treatment beams, is between 350 and 850 nm. For example, visible light 68 may be red, with a wavelength of 600-700 nm, while the treatment beams may be green, with a wavelength of 527-537 nm.

Typically, the optical unit comprises an optical bench, and at least some of the aforementioned optical components belonging to the optical unit, such as the radiation source, the galvo mirrors, and the beam combiner, are coupled to the optical bench. Typically, the optical unit further comprises a front face 33, through which the treatment beams and visible light 68 pass. For example, optical unit 30 may comprise an encasement 31, which at least partially encases the optical bench and comprises front face 33. (Encasement 31 may be made of a plastic, a metal, and/or any other suitable material.) Alternatively, front face 33 may be attached to, or may be an integral part of, the optical bench.

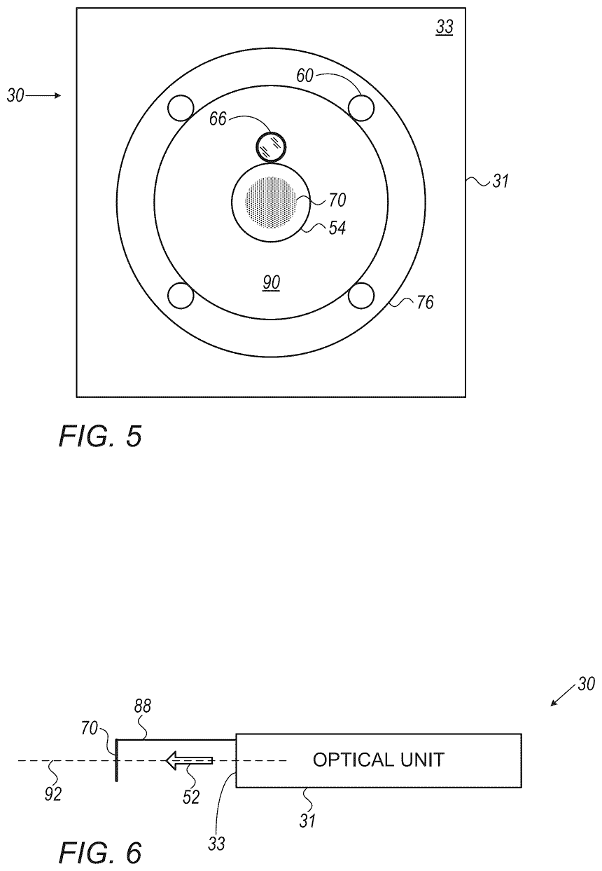

In some embodiments, front face 33 is shaped to define an opening 58, through which the treatment beams and visible light 68 pass. In other embodiments, as shown in FIG. 5 (described below), the front face comprises an exit window in lieu of opening 58, such that visible light 68 and treatment beams 52 pass through the exit window. The exit window may be made of a plastic, a glass, or any other suitable material.

Typically, optical unit 30 further comprises one or more illumination sources 60 comprising, for example, one or more LEDs, such as white-light or infrared LEDs. For example, the optical unit may comprise a ring of LEDs surrounding opening 58. In such embodiments, controller 44 may cause illumination sources 60 to intermittently flash light at the eye, as described in International Patent Application PCT/IB2019/055564, whose disclosure is incorporated herein by reference. This flashing may facilitate the imaging performed by the camera, and may further help constrict the pupil of the eye. (For ease of illustration, the electrical connection between controller 44 and illumination sources 60 is not shown explicitly in FIG. 2.) In some embodiments, illumination sources 60 are coupled to front face 33, as shown in FIG. 2.

To facilitate positioning the optical unit, the optical unit may comprise a plurality of beam emitters 62 (comprising, for example, respective laser diodes), which are configured to shine a plurality of triangulating range-finding beams on the eye, e.g., as described in International Patent Application PCT/IB2019/055564. In some embodiments, beam emitters 62 are coupled to front face 33, as shown in FIG. 2. In other embodiments, beam emitters 62 are coupled directly to the optical bench.

Optical unit 30 is mounted onto an XYZ stage unit 32, which is controlled by a control mechanism 36, such as a joystick. Using control mechanism 36, the user of system 20 may position the optical unit (e.g., by adjusting the distance of the optical unit from the eye) prior to treating the eye. In some embodiments, XYZ stage unit 32 comprises locking elements configured to inhibit motion of the stage unit following the positioning of the stage unit.

In some embodiments, XYZ stage unit 32 comprises one or more motors 34, and control mechanism 36 is connected to interface circuitry 46. As the user manipulates the control mechanism, interface circuitry 46 translates this activity into appropriate electronic signals, and outputs these signals to controller 44. In response to the signals, the controller controls the motors of the XYZ stage unit.

In other embodiments, XYZ stage unit 32 is controlled manually by manipulating the control mechanism. In such embodiments, the XYZ stage unit may comprise a set of gears instead of motors 34.

System 20 further comprises a headrest 24, comprising a forehead rest 26 and a chinrest 28. During the trabeculoplasty procedure, patient 22 presses his forehead against forehead rest 26 while resting his chin on chinrest 28. In some embodiments, headrest 24 further comprises an immobilization strap 27, configured to secure the patient's head from behind and thus keep the patient's head pressed against the headrest.

In some embodiments, as shown in FIG. 1, headrest 24 and XYZ stage unit 32 are both mounted onto a surface 38, such as a tray or tabletop. (In some such embodiments, the headrest is L-shaped, and is attached to the side, rather than the top, of surface 38.) In other embodiments, the XYZ stage unit is mounted onto surface 38, and the headrest is attached to the XYZ stage unit.

Typically, as shown in FIG. 1, while irradiating the patient's eye, the optical unit is directed obliquely upward toward the eye while the eye gazes obliquely downward toward the optical unit, such that optical path 92 is oblique. For example, the optical path may be oriented at an angle θ of between five and twenty degrees with respect to the horizontal. Advantageously, this orientation reduces occlusion of the patient's eye by the patient's upper eyelid and associated anatomy.

In some embodiments, as shown in FIG. 1, the oblique orientation of the optical path is achieved by virtue of the optical unit being mounted on a wedge 40, which is mounted on the XYZ stage unit. In other words, the optical unit is mounted onto the XYZ stage unit via wedge 40. (Wedge 40 is omitted from FIG. 2.)

System 20 further comprises a monitor 42, configured to display the images of the eye acquired by the camera. Monitor 42 may be attached to optical unit 30 or disposed at any other suitable location, such as on surface 38 next to device 21. In some embodiments, monitor 42 comprises a touch screen, and the user inputs commands to the system via the touch screen. Alternatively or additionally, system 20 may comprise any other suitable input devices, such as a keyboard or a mouse, which may be used by the user.

In some embodiments, monitor 42 is connected directly to controller 44 over a wired or wireless communication interface. In other embodiments, monitor 42 is connected to controller 44 via an external processor, such as a processor belonging to a standard desktop computer.

In some embodiments, as shown in FIG. 2, controller 44 is disposed within XYZ stage unit 32. In other embodiments, controller 44 is disposed externally to the XYZ stage unit. Alternatively or additionally, the controller may cooperatively perform at least some of the functionality described herein with another, external processor.

In some embodiments, at least some of the functionality of controller 44, as described herein, is implemented in hardware, e.g., using one or more Application-Specific Integrated Circuits (ASICs) or Field-Programmable Gate Arrays (FPGAs). Alternatively or additionally, controller 44 may perform at least some of the functionality described herein by executing software and/or firmware code. For example, controller 44 may comprise a central processing unit (CPU) and random access memory (RAM). Program code, including software programs, and/or data may be loaded into the RAM for execution and processing by the CPU. The program code and/or data may be downloaded to the controller in electronic form, over a network, for example. Alternatively or additionally, the program code and/or data may be provided and/or stored on non-transitory tangible media, such as magnetic, optical, or electronic memory. Such program code and/or data, when provided to the controller, produce a machine or special-purpose computer, configured to perform the tasks described herein. In some embodiments, the controller comprises a system on module (SOM), such as the Varisite™ DART-MX8M.

### The Optical Filter

As shown in FIG. 2, trabeculoplasty device 21 further comprises an optical filter 70, which is opaque to the treatment beams but not to visible light 68. (In some embodiments, the optical filter is also transparent to the light emitted by illumination sources 60 and/or the light emitted by beam emitters 62.) Optical filter 70 is configured to inhibit passage of the treatment beams, but not visible light 68, therethrough, while interposing between the beam-directing elements and the pupil of eye 25. (For ease of description, this act of interposing is referred to as “covering” the pupil.) Thus, the optical filter protects the retina of the eye while allowing the eye to fixate on fixation target 64. Typically, the optical filter is elliptical, e.g., circular. The optical filter may attenuate the treatment beams with an optical density (OD) that is between one and six, or even greater than six.

In some embodiments, optical filter 70 inhibits the passage of the treatment beams by absorbing the treatment beams. For example, optical filter 70 may comprise an absorptive plastic and/or an absorptive glass, such as a material used in the Thorlabs LG14 laser safety glasses or the Laser Safety Industries 35-235 laser safety glasses.

In such embodiments, the optical filter may comprise an anti-reflective surface configured to reduce reflection of the treatment beams. For example, the optical filter may comprise an absorptive material having a surface that is etched (e.g., subwavelength surface textured) or coated (e.g., with a dielectric coating) to reduce reflections therefrom. (The aforementioned coating may comprise a single layer or multiple layers.) In some embodiments, both surfaces of the optical filter are anti-reflective; the front anti-reflective surface, which faces the optical unit, protects the user of the system from any primary reflections, while the back anti-reflective surface protects the eye from any secondary reflections. In other embodiments, only one of the surfaces, such as the front surface, is anti-reflective.

Alternatively or additionally to absorbing the treatment beams, the optical filter may inhibit the passage of the treatment beams by reflecting the treatment beams. For example, the optical filter may comprise a piece of transparent material (such as plastic or glass, for example) coated with a thin-film or broadband reflective coating. Such a coating may be applied to the front surface of the piece of material, which faces the optical unit, and/or the back surface, which faces the eye.

In some embodiments, the trabeculoplasty device further comprises a contact optic 72, which comprises the optical filter and is configured to contact the eye. (Typically, contact optic 72 is elliptical, e.g., circular.) During the procedure, contact optic 72 contacts eye 25, with optical filter 70 covering the pupil of the eye. (It is noted that in the context of the present application, including the claims, the term “contact” may include contact via an optical coupling fluid or a gel.) Typically, the contact optic is curved, so as to conform to the shape of the eyeball of eye 25. In some embodiments, the thickness of the contact optic is less than 2 mm, such that the contact optic may be worn in the eye without being held in place.

In some embodiments, contact optic 72 comprises the optical filter along with a transparent portion 74, which surrounds the optical filter and is transparent to the treatment beams and to visible light 68. (Typically, transparent portion 74 is annular-elliptical, e.g., annular-circular.) In such embodiments, the diameter d1 of the contact optic is typically 12-17 mm, with the diameter d2 of the optical filter typically being less than 10 mm. During the procedure, optical filter 70 covers the pupil of the eye (and, typically, at least part of the cornea surrounding the pupil), while transparent portion 74 covers the targeted vicinity of the limbus. Hence, the vicinity of the limbus may be irradiated by the treatment beams and may also be imaged by camera 54.

For example, contact optic 72 may comprise a transparent optic having a central portion coated with a reflective coating, which constitutes the optical filter, along with an uncoated peripheral portion. Alternatively, for embodiments in which the optical filter comprises an absorptive material, the optical filter may be embedded in the center of a transparent optic. Such embedding may be performed using diffusion bonding, welding, soldering, or an adhesive.

In some embodiments, the contact optic further comprises another optical filter that surrounds the transparent portion and inhibits passage of the treatment beams therethrough, as described below with reference to FIGS. 4A-B.

In other embodiments, the contact optic comprises the optical filter without comprising transparent portion 74. In such embodiments, diameter d1 (which is equal to diameter d2 due to the absence of transparent portion 74) is typically less than 10 mm, such that, during the procedure, the limbus and/or sclera of the eye are exposed. Although corrective contact lenses typically have a larger diameter so as not to discomfort the wearer, the inventors have realized that contact optic 72 is unlikely to discomfort the patient despite its small size, particularly if anesthetic eye drops are applied to the eye before the contact optic is placed in the eye.

In some embodiments, the trabeculoplasty device further comprises an eye-stabilizing structure 76, through which the various types of radiation described herein pass. Typically, the length of structure 76 is between 10 and 70 mm.

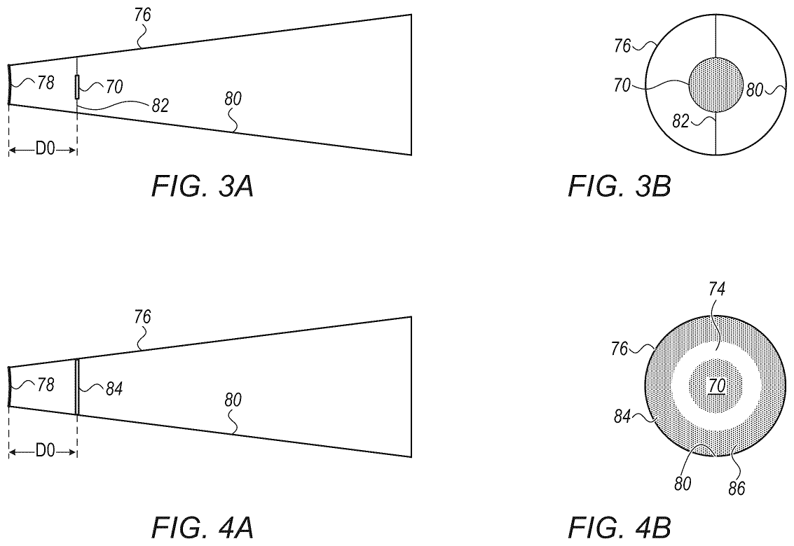

In such embodiments, optical filter 70 is typically coupled to structure 76. For example, as shown in FIG. 2, contact optic 72 may be coupled to the distal end of the structure, such that optical filter 70 is coupled to the distal end of structure 76 by virtue of the contact optic comprising the optical filter. (In such embodiments, the thickness of the contact optic is typically greater than 2 mm; for example, the thickness may be between 3 and 20 mm.) For example, the edge of the contact optic may be coupled to the distal end of the structure using a mechanical attachment mechanism, such as a screw, and/or any suitable adhesive. Alternatively, optical filter 70 may be coupled to structure 76 in other ways, as further described below with reference to FIGS. 3A-B and 4A-B.

Typically, structure 76 is hollow, such that the radiation described herein passes through air within the structure. For example, structure 76 may comprise a frustum-shaped or cylindrically-shaped tube, which may be made from metal, glass, or any other suitable material. In some such embodiments, the structure comprises a continuous wall. In other such embodiments, structure 76 is shaped to define one or more openings. For example, structure 76 may comprise a wire structure, such as a wire mesh. As further described below with reference to FIG. 8, an advantage of such embodiments is that the user may view the eye through the openings.

In other embodiments, structure 76 comprises a frustum-shaped or cylindrically-shaped transparent piece of material (e.g., glass), such that the radiation passes through the material.

Structure 76 typically provides several advantages, alternatively or additionally to holding optical filter 70. For example, as shown in FIG. 1, the distal end of structure 76 may retract the eyelids of eye 25. Additionally, the distal end of the structure may stabilize the eye, i.e., inhibit the eye from moving. Furthermore, structure 76 may facilitate placing the optical unit at the correct distance from the patient. Moreover, the inner and/or outer surface of structure 76 may be configured to absorb any misaimed treatment beams or scattered light, and/or to block any external light from interfering with the camera. For example, the inner and/or outer surface of the structure may comprise black flat paint, black flocked paper, or Vantablack.

In some embodiments, as shown in FIGS. 1-2, the proximal end of the structure is configured to couple to the optical unit, e.g., via a spring. In such embodiments, the trabeculoplasty device may further comprise a pressure sensor coupled to structure 76 and configured to measure the pressure applied to the structure. Based on the pressure measurement, the user may position the optical unit such that the pressure applied to the structure, which is equivalent to the pressure applied to the eye, is within a predefined range suitable for stabilizing the eye without hurting the patient. In other embodiments, the structure is not coupled to the optical unit, but rather, is held by the user of system 20 during the procedure.

In some embodiments, structure 76 is disposable, and a different respective structure is used for each patient.

As noted above, the optical unit may comprise an exit window in lieu of opening 58. In such embodiments, as further described below with reference to FIG. 5, the optical filter may overlay, or may be embedded within, the exit window.

As further described below with reference to FIG. 8, during the procedure, controller 44 may process images of the eye to calculate the position of each of the target regions. In some embodiments, the controller identifies contact optic 72 in each of the images, and verifies that the contact optic is in the correct location relative to the eye. Optionally, the controller may also calculate the position of the target region with reference to the position of the contact optic.

In some embodiments, to facilitate identifying the contact optic, the contact optic is opaque to wavelengths to which the camera is sensitive. For example, for embodiments in which the wavelength of the fixation light is between 600 and 700 nm, the camera may be sensitive only to wavelengths below 600 nm, and the contact optic may be opaque to wavelengths below 600 nm.

In other embodiments, the controller does not identify the contact optic in any of the images.

In some embodiments—particularly if structure 76 is not used—a finger, a speculum, or another tool may be used to retract one or both of the eyelids of eye 25.

It is emphasized that the configuration of device 21 shown in FIG. 2 is provided by way of example only. Moreover, alternatively or additionally to the components shown in FIG. 2, device 21 may comprise any suitable components.

Reference is now made to FIGS. 3A-B, which are schematic illustrations of structure 76 as viewed from the side and from the front, respectively, in accordance with some embodiments of the present invention. Reference is further made to FIGS. 4A-B, which are also schematic illustrations of structure 76 as viewed from the side and from the front, respectively, in accordance with some embodiments of the present invention.

In some embodiments, the optical filter is situated proximally to the distal end of eye-stabilizing structure 76, such that the distal end of the eye-stabilizing structure, but not the optical filter, contacts the eye. For example, as shown in FIGS. 3A and 4A, the optical filter may be mounted to the inner wall 80 of structure 76, e.g., at a distance D0 of 0.5-20 mm from the distal end of the structure. In such embodiments, diameter d2 of the optical filter (FIG. 2) is matched to distance D0, such that the optical filter covers the pupil without covering the targeted regions of the eye. In other words, for a smaller D0, d2 is made larger, and vice versa.

In some embodiments, as shown in FIGS. 3A-B, the optical filter is mounted to inner wall 80 via one or more longitudinal elements 82, such as stiff wires or posts, extending between the inner wall and the optical filter.

In other embodiments, as shown in FIGS. 4A-B, the trabeculoplasty device comprises an optic 84, which comprises optical filter 70 along with transparent portion 74. In such embodiments, the edge of optic 84 may be coupled to inner wall 80 using a mechanical attachment mechanism, such as a screw, and/or any suitable adhesive. (Since optic 84 does not need to conform to the shape of the eye, optic 84 is typically not curved.)

In some embodiments, optic 84 further comprises another optical filter 86 that surrounds transparent portion 74 and is configured to inhibit the passage of the treatment beams. (Typically, optical filter 86 is annular-elliptical, e.g., annular-circular.) For example, optical filter 86 may comprise an absorptive material and/or a thin-film reflective coating, such that optical filter 86 absorbs and/or reflects the treatment beams. Typically, optical filter 86 comprises the same materials as does optical filter 70, and/or is etched in the same way as is optical filter 70.

For embodiments in which the optical filter is situated proximally to the distal end of the eye-stabilizing structure, the distal end of the structure may comprise a contact optic 78 configured to help stabilize the eye by contacting the eye. Typically, contact optic 78, or at least the portion of the contact optic that covers the treated vicinity of the limbus, is transparent to the treatment beams and to visible light. Typically, the diameter of contact optic 78 is between 12 and 17 mm. (It is noted that the view of the eye-stabilizing structure in each of FIGS. 3B and 4B corresponds to the view seen through contact optic 78.)

In some embodiments, contact optic 78 is shaped to define an opening, and the trabeculoplasty device further comprises (i) a suction-applying device, such as a pump, and (ii) a tube having a proximal end connected to the suction-applying device and a distal end passing through the opening. In such embodiments, the suction-applying device may apply a suction force through the tube, thus helping the contact optic to stabilize the eye.

Alternatively or additionally to contact optic 78, the distal end of the structure may comprise a contact ring configured to help stabilize the eye by contacting the eye. Typically, the contact ring contacts the eye outside of, and sufficiently far from (e.g., at least 2 mm from), the limbus, such that the portion of the eye targeted by the treatment beams, which typically includes the limbus and/or part of the sclera, is exposed to the treatment beams and to the camera. For example, the diameter of the contact ring may be between 14 and 18 mm. In some embodiments, the contact ring does not contact the eye continuously, but rather, only over several (e.g., between three and ten) points of contact.

In some embodiments, to facilitate stabilizing the eye, suction may be applied through the contact ring, as described above for contact optic 78. In addition to stabilizing the eye, the contact ring may retract the eyelids of the eye.

Reference is now made to FIGS. 5-6, which are schematic illustrations of optical filter 70 coupled to optical unit 30, in accordance with some embodiments of the present invention.

In some embodiments, optical filter 70 is coupled to the optical unit.

For example, as shown in the frontal view of the optical unit in FIG. 5, the optical filter may overlay, or may be embedded within, an exit window 90 belonging to front face 33. For example, the optical filter may comprise a thin-film reflective coating that coats exit window 90. (To avoid any confusion, it is noted that the frontal view of the optical unit shown in FIG. 5 is not the view typically seen by the patient during the procedure; in the latter view, optical filter 70 fills the patient's field of view, given that the optical filter typically covers the patient's entire pupil.)

As another example, as shown in FIG. 6, a longitudinal element 88, such as a stiff wire or a post, may extend between the front face and the optical filter, and the optical filter may be coupled to the front face via longitudinal element 88. In other words, the proximal end of longitudinal element 88 may be coupled to front face 33 (e.g., to exit window 90), with the distal end of the longitudinal element coupled to the optical filter. (For embodiments in which eye-stabilizing structure 76 (FIG. 1) is used, longitudinal element 88 typically passes through the eye-stabilizing structure.) Typically, the longitudinal element is parallel to optical path 92.

Typically, the distance between the front face of the optical unit and the optical filter, which is generally equal to the length of longitudinal element 88, is between 10 and 50 mm, and/or the distance between the optical filter and the eye is between 0.1 and 20 mm.

As yet another alternative, the optical filter may be coupled to the optical bench belonging to the optical unit. For example, the optical filter may be coupled to the optical bench downstream from the most downstream of the beam-directing elements, such as between beam combiner 56 and opening 58 (FIG. 2), such that the optical filter interposes between the most downstream of the beam-directing elements and the pupil of the eye.

### Alternative Embodiments

In alternative embodiments, optical filter 70 is opaque both to the treatment beams and to the fixation light, i.e., the visible light transmitted by light source 66 (FIG. 2). For example, the optical filter may comprise a metal or ceramic disk. Although, in such embodiments, the optical filter inhibits both the visible light and the treatment beams from reaching the pupil of eye 25, eye 25 may nonetheless be oriented for treatment, as described immediately below. Hence, the optical filter may be manufactured more cheaply and easily, without compromising the safety and efficacy of the procedure.

For example, to allow eye 25 to fixate on the light source, the light source may be coupled to the optical bench parallel to or downstream from the optical filter. For example, the optical filter and the light source may each be coupled to the optical bench between the most downstream of the beam-directing elements and opening 58, such that (i) the optical filter interposes between the most downstream of the beam-directing elements and the pupil of the eye, and (ii) light source 66 is disposed next to optical path 92, parallel to or downstream from the optical filter. Alternatively, the light source may be disposed behind the optical filter, as in FIG. 2; however, one or more reflectors coupled to the optical bench may reflect the fixation light through opening 58, such that the fixation light may reach eye 25 without needing to pass through the optical filter.

Alternatively, for embodiments in which eye 25 cannot see the fixation light through the optical filter, eye 25 may be oriented by causing the untreated eye of the patient to fixate on light source 66. In such embodiments, prior to the procedure, the patient is instructed to fixate the untreated eye on the light source. Subsequently, by virtue of the light source transmitting visible light, the untreated eye fixates on the light source, such that eye 25, by virtue of moving in tandem with the untreated eye, gazes approximately along optical path 92. Thus, while the untreated eye fixates on the fixation target, the treated eye may be irradiated with the treatment beams.

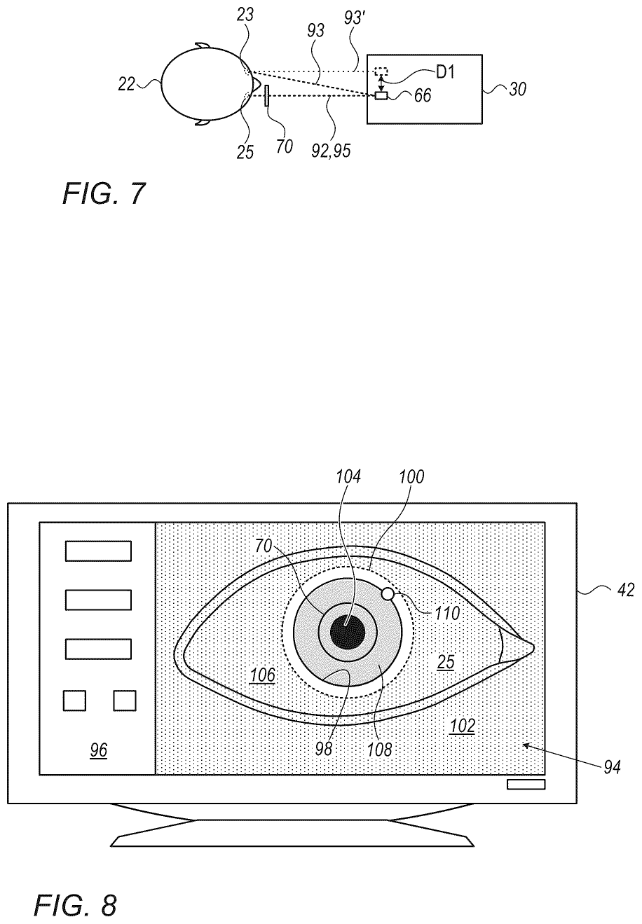

In this regard, reference is now made to FIG. 7, which is a schematic illustration of an alternative technique for orienting eye 25 for treatment, in accordance with some embodiments of the present invention. (FIG. 7 shows a schematic overhead view of optical unit 30 and patient 22.)

In some embodiments, the position of the light source is adjustable. For example, the light source may be mounted on a slider, such that the distance of the light source from optical path 92 is variable. In such embodiments, to facilitate orienting eye 25 via the fixation of the untreated eye 23 of the patient on the light source, the position of the light source is varied responsively to the degree to which the patient's eyes can converge onto the light source.

For example, for those patients, such as younger patients, whose eyes can converge onto light source, the light source may be aligned, at least approximately, with optical path 92, as described above with reference to FIG. 2. As untreated eye 23 fixates on the light source along a first line-of-sight 93, eye 25 gazes toward the light source along a second line-of-sight 95, which is approximately coincident with optical path 92.

For other patients, particularly older patients, whose eyes cannot converge onto the light source due to the relatively small distance between the patient and the light source, the light source may be displaced from the optical path towards the untreated eye, typically along an axis parallel to the interpupillary axis of the patient. For example, the light source may be placed at a distance D1 from the optical path that is approximately equal to the interpupillary distance of the patient; thus, for example, D1 may be between 45 and 80 mm. During the procedure, the untreated eye fixates on the light source along an alternate first line-of-sight 93′, such that second line-of-sight 95 is approximately coincident with optical path 92.

In other embodiments, a single position of the light source is used for all patients. For example, the light source may be displaced from the optical path towards the untreated eye, typically along an axis parallel to the interpupillary axis of the patient, by a distance that is less than, such as 30%-70% of, an average or median interpupillary distance for a suitable population of patients. For example, assuming an average interpupillary distance of 62 mm, the light source may be displaced from the optical path by between 18 and 44 mm. Advantageously, this position may help second line-of-sight 95 to be sufficiently coincident with optical path 92, regardless of the degree to which the patient's eyes converge onto the light source.

### Protecting the Eye During Treatment

Reference is now made to FIG. 8, which is a schematic illustration of an image 94 of eye 25 treated in accordance with some embodiments of the present invention.

Prior to the procedure, the camera acquires image 94. Based on the image, the user defines one or more target regions 110 of the eye that are to be irradiated. Typically, as described in International Patent Application PCT/IB2019/055564, target regions 110 are defined semi-automatically, based on input from the user via a graphical user interface (GUI) 96 displayed on monitor 42. Typically, at least part of each target region is located near (e.g., within 2 mm of) the limbus 98 of the eye; for example, each target region may overlap limbus 98.

Typically, following the definition of the target regions, the controller executes a mock trabeculoplasty by causing radiation source 48 (FIG. 2) to sweep an aiming beam across the target regions. Alternatively, the aiming beam may be emitted by an additional radiation source, such as a lower-power laser, that is collinear with radiation source 48. (Typically, the aiming beam differs from the treatment beams at least in that the energy of the aiming beam is subtherapeutic.) While this sweep is performed, camera 54 (FIG. 2) acquires images of the eye at a relatively high frequency and the images are displayed on monitor 42, such that the user may validate the target regions. Alternatively or additionally, for embodiments in which structure 76 (FIG. 1) does not have a continuous wall, the user may view the aiming beam, as it impinges on the eye, through an opening in the structure.

Following the validation of the target regions, the user starts the procedure, typically by entering an appropriate input via GUI 96. Subsequently, as described above with reference to FIG. 1, the target regions of the eye are irradiated by radiation source 48, i.e., the radiation source fires respective treatment beams at the target regions.

In some embodiments, during the procedure, the camera acquires images of the eye at a relatively high frequency (e.g., at a frequency greater than 40 Hz or 50 Hz). Based on the images, controller 44, by executing image-processing software, tracks motion of the eye, e.g., by identifying the center of the limbus in each image. Based on the motion tracking, the controller, prior to the irradiation of each target region, locates the target region in the FOV of the camera, and then controls the beam-directing elements such that a treatment beam impinges on the target region. In other embodiments, the eye-stabilizing structure may sufficiently stabilize the eye such as to obviate the need for high-frequency motion tracking. In such embodiments, during the procedure, the camera may acquire images of the eye, and the controller may check for movement of the eye, at a lower frequency; alternatively, the image acquisition and motion tracking may be omitted entirely. Similarly, high-frequency motion tracking may be omitted if only a small number of treatment beams are fired. For example, the radiation source may emit a single treatment beam—shaped, for example, as an ellipse or an arc—that simultaneously irradiates all the target regions.

In any case, regardless of whether high-frequency motion tracking is performed, embodiments of the present invention, as an extra precaution, protect the eye from any (unlikely) misaimed treatment beams that deviate too far from the limbus. Such protection may be effected using optical filter 70, which was described at length above. In particular, optical filter 70 may protect the retina of the eye by covering the pupil 104 of the eye. As described above with reference to FIG. 2, optical filter 70 may also cover at least part of the cornea 108 surrounding the pupil, thus protecting cornea 108. Alternatively or additionally, a peripheral optical filter, such as optical filter 86 belonging to optic 84 (described above with reference to FIGS. 4A-B), may protect the sclera 106 of the eye by covering sclera 106.

In some embodiments, the optical filter is opaque to the camera (i.e., to the frequencies to which the camera is sensitive). In such embodiments, the controller may identify the optical filter in the images acquired by the camera, and may further verify that the optical filter is covering the pupil, e.g., as described above with reference to FIG. 2. In other embodiments, the optical filter is transparent to the camera. (FIG. 8 assumes that the optical filter is transparent to the camera; nevertheless, for clarity, the edge of the optical filter is shown in FIG. 8.)

In some embodiments, alternatively or additionally to using an optical filter, the eye is protected using a virtual filter, implemented in the software and/or firmware run by the controller. In particular, prior to the firing of any treatment beams, controller 44 (FIG. 1) causes camera 54 to acquire image 94. Subsequently to the acquisition of image 94, the controller, based on the image, identifies one or more static regions in the FOV of the camera that include non-targeted portions of the eye. Subsequently, prior to the irradiation of each target region of the eye (i.e., prior to the firing of a treatment beam at the target region), the controller ascertains that the target region is not within any of the static regions. In response to ascertaining that the target region is not within any of the static regions, the controller causes the radiation source to irradiate the target region; otherwise, the treatment beam is not fired. Furthermore, in some embodiments, the controller inhibits the beam-directing elements from being aimed at any of the static regions even while the radiation source is inactive.

For example, as described in International Patent Application PCT/IB2019/055564, based on image 94, the controller may identify a central static region encompassing pupil 104 in the image. Subsequently, the controller may inhibit the radiation source from firing any treatment beams at the central static region. The central static region may thus protect the retina of the eye in the event that optical filter 70 is not used, or provide extra protection to that provided by the optical filter.

Alternatively or additionally, the controller may identify a peripheral static region 102 that, in the image, is outside limbus 98 of the eye. Static region 102 may protect sclera 106 in the event that a peripheral optical filter is not used, or provide extra protection to that provided by a peripheral optical filter. As shown in FIG. 8, the peripheral static region may further protect the eyelids of the eye, along with other nearby tissue.

Typically, the peripheral static region includes the portion of the camera's FOV lying outside an elliptical (e.g., circular) boundary 100, which, in image 94, may be located outside limbus 98 at a distance of between 1 and 5 mm from the limbus.

It is emphasized that each of the static regions is “static” by virtue of being defined in terms of the FOV of the camera, such that the position of the region is not adjusted by the controller even in response to detected motion of the eye. Thus, the eye is protected even in the event of an (unlikely) error in the motion tracking performed by the controller.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A system, comprising:
a camera, configured to acquire an image of an eye of a patient;
a radiation source; and
a controller, configured to:
identify a static region in a field of view of the camera that, in the image, is outside a limbus of the eye, and
treat one or more target regions of the eye, by, for each target region:
ascertaining that the target region is not within the static region, and
in response to the ascertaining, causing the radiation source to irradiate the target region.

2. The system according to claim 1, wherein the static region is a central static region encompassing a pupil of the eye.

3. The system according to claim 1, wherein the static region is a peripheral static region outside a limbus of the eye.

4. The system according to claim 1, wherein the static region has an elliptical boundary.

5. The system according to claim 1, further comprising:
a light source; and
one or more beam-directing elements,
wherein the radiation source is configured to irradiate the eye with one or more treatment beams by emitting the treatment beams toward the beam-directing elements, while the eye fixates on the light source by virtue of the light source transmitting visible light.

6. The system according to claim 5, further comprising an optical filter configured to inhibit passage of the treatment beams, but not the visible light, therethrough, while interposing between the beam-directing elements and a pupil of the eye.

7. The system according to claim 5, wherein the radiation source is configured to irradiate a first eye of a patient with the one or more treatment beams by emitting the treatment beams toward the beam-directing elements such that the beam-directing elements direct the treatment beams along an optical path to the first eye, and
wherein the system comprises an optical filter configured to inhibit the visible light and the treatment beams from reaching a pupil of the first eye,
the light source being displaced from the optical path towards a second eye of the patient, and
the radiation source being configured to irradiate the first eye while the second eye fixates on the light source by virtue of the light source transmitting the visible light.

8. The system according to claim 7, wherein the light source is displaced from the optical path by 18-44 mm.

9. The system according to claim 1, further comprising an eye-stabilizing structure, wherein the radiation source is configured to irradiate the eye by emitting one or more treatment beams through the structure.

10. A method, comprising:
using a camera, acquiring an image of an eye of a patient;
identifying a static region in a field of view of the camera that, in the image, is outside a limbus of the eye; and
treating one or more target regions of the eye, by, for each target region:
ascertaining that the target region is not within the static region, and
in response to the ascertaining, irradiating the target region.

11. The method according to claim 10, wherein the static region is a central static region encompassing a pupil of the eye.

12. The method according to claim 10, wherein the static region is a peripheral static region outside a limbus of the eye.

13. The method according to claim 10, wherein the static region has an elliptical boundary.

14. The method according to claim 10, further comprising:
transmitting visible light from a light source toward one or more beam-directing elements,
wherein treating the one or more target regions comprises, while the eye fixates on the visible light, irradiating the eye with one or more treatment beams emitting from a radiation source toward the beam-directing elements.

15. The method according to claim 14, further comprising interposing an optical filter to inhibit passage of the treatment beams, but not the visible light, therethrough between the beam-directing elements and a pupil of the eye.

16. The method according to claim 14, wherein irradiating the eye comprises irradiating a first eye of a patient with the one or more treatment beams by emitting the treatment beams toward the beam-directing elements such that the one or more beam-directing elements direct the treatment beams along an optical path to the first eye while a second eye of the patient fixates on the visible light, and
wherein the method comprises interposing an optical filter to inhibit the visible light and the treatment beams between the one or more beam-directing elements and a pupil of the first eye.

17. The method according to claim 16, wherein the one or more beam-directing elements direct the treatment beams along an optical path to the first eye, and wherein the method further comprises, prior to irradiating the first eye, displacing the light source from the optical path toward the second eye.

18. The system according to claim 10, wherein treating the one or more target regions comprises emitting one or more treatment beams toward the eye through an eye-stabilizing structure.
